(19) United States
(12) Patent Application Publication
(10) Pub. No.: US 2017/0274005 A1
(43) Pub. Date: Sep. 28, 2017

(54) TREATMENT OF RETINITIS PIGMENTOSA USING HYALURONIC ACID-CARBON NANOMATERIAL-PHOTOSENSITIZER COMPLEX

(71) Applicant: POSTECH ACADEMY-INDUSTRY FOUNDATION, Pohang (KR)

(72) Inventors: Sei Kwang HAHN, Pohang (KR); Songeun Beack, Jeonju (KR); Jun-Sub Choi, Yongin (KR); Choun-Ki Joo, Seoul (KR)

(21) Appl. No.: 15/384,434

(22) Filed: Dec. 20, 2016

(30) Foreign Application Priority Data
Mar. 22, 2016 (KR) 10-2016-0034274

(51) Int. Cl. A61K 31/728; A61K 41/00; A61K 9/00; A61K 33/44

(52) U.S. Cl. CPC A61K 31/728; A61K 33/44; A61K 41/0071; A61K 9/0019; A61K 9/0048; A61K 47/481; A61K 47/48076

(57) ABSTRACT

Disclosed is a complex for remedying or treating a retinal disease. The complex includes a hyaluronic acid or a derivative thereof, a carbon nanomaterial covalently bonded to the hyaluronic acid or the derivative thereof, and a photosensitizer bonded to the carbon nanomaterial. The complex for remedying or treating the retinal disease according to the present invention includes a hyaluronic acid-carbon nanomaterial-photosensitizer complex, thus selectively preventing the formation of active oxygen in retinal pigment epithelium cells for a relatively long period of time. Accordingly, the complex has excellent therapeutic efficacy and easily infiltrates into cells. Further, the present invention provides a composition for remedying or treating a retinal disease including the hyaluronic acid-carbon nanomaterial-photosensitizer complex.

### BACKGROUND OF THE INVENTION

1. Field of the Invention

The present invention relates to a complex for remedying or treating a retinal disease and a composition for remedying or treating a retinal disease including the same. More particularly, the present invention relates to a complex for remedying or treating a retinal disease, which includes a hyaluronic acid-carbon nanomaterial-photosensitizer complex to thus prevent active oxygen from being formed, and a composition for remedying or treating a retinal disease including the same.

2. Description of the Related Art

Retinitis pigmentosa (RP) is a disease affecting vision due to trouble in the photoreceptor of the retina. Representative examples of this disease include retinal degeneration diseases that affect the retinal pigment epithelial cells and photoreceptors. Examples of representative symptoms thereof include nyctalopia, contraction of visual field, and visual impairment. Effective methods of treating retinitis pigmentosa are still not known, but research into various treatment methods has been made. Representative examples of such treatment methods include gene therapy, retinal transplantation, electrical stimulation, and active oxygen prevention.

A photosensitizer has been used in skin or eye treatment and in photodynamic therapy. When a photosensitizer is irradiated with light, oxygen in the surrounding tissue is changed from triplet oxygen to singlet oxygen to form active oxygen, but active oxygen affects the degeneration of retinal cells. Further, since the photosensitizer remains in the skin and eyes for a long period of time after treatment, the photosensitizer exhibits a photosensitivity-related side effect of killing the normal cells of the skin or eyes when a patient is exposed to light. Therefore, the patient has the inconvenience of having to stay in a dark room for a long period of six weeks or more after treatment.

When the photosensitizer is reformed so that the photosensitizer has hydrophilicity to reduce the non-specific accumulation in normal tissue to thereby solve the problem of the remaining photosensitizer, there is a merit in that the photosensitizer is rapidly released from the human body. However, there are drawbacks in that the amount of the photosensitizer that is administered must be increased in order to deliver the photosensitizer in an amount sufficient for treatment and in that therapeutic efficacy is low because the photosensitizer does not readily infiltrate into cells.

### SUMMARY OF THE INVENTION

Accordingly, the present invention has been made keeping in mind the above problems occurring in the related art, and an object of the present invention is to provide a complex that is used to remedy or treat a retinal disease, has excellent therapeutic efficacy, and easily infiltrates into cells. The complex includes a hyaluronic acid-carbon nanomaterial-photosensitizer complex, thus selectively preventing the formation of active oxygen in retinal pigment epithelium cells over a relatively long period of time.

Another object of the present invention is to provide a composition for remedying or treating a retinal disease including the hyaluronic acid-carbon nanomaterial-photosensitizer complex.

In order to accomplish the above objects, the present invention provides a complex for remedying or treating a retinal disease, including a hyaluronic acid or a derivative thereof, a carbon nanomaterial covalently bonded to the hyaluronic acid or the derivative thereof, and a photosensitizer bonded to the carbon nanomaterial.

The complex for remedying or treating the retinal disease may prevent active oxygen from being formed.

The complex for remedying or treating the retinal disease may prevent active oxygen from being formed in a retinal pigment epithelium cell or a retinal vessel.

The retinal disease may include one or more selected from retinitis pigmentosa, dry macular degeneration, and nyctalopia.

Treatment using the complex for remedying or treating the retinal disease may be performed under a lightless condition.

The complex for remedying or treating the retinal disease may be administered using one or more selected from intravitreous injection, administration of eye drops, and intravenous injection.

The derivative of the hyaluronic acid may be a hyaluronic acid substituted with cystamine having the structure of the following Chemical Formula 1.

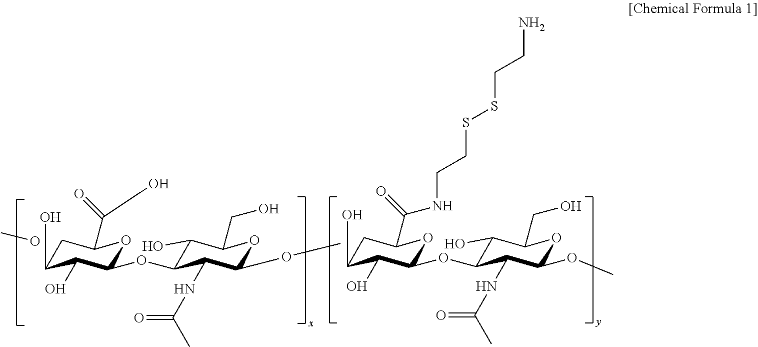

In Chemical Formula 1, x and y are each independently an integer within the range from 16 to 2,500.

Substitution with cystamine may be performed at a substitution ratio of 10 to 30% based on the amount of hyaluronic acid.

The carbon nanomaterial may include one or more selected from a carbon quantum dot, a carbon nanotube, graphene oxide, graphene, and fullerene.

The photosensitizer may include one or more selected from chlorine e6 (Ce6), a porphyrin-based photosensitizer, and a non-porphyrin-based photosensitizer.

The carbon nanomaterial may be the carbon quantum dot and the photosensitizer may be the Ce6.

1 to 2 parts by weight of the Ce6 may be bonded to 1 part by weight of the carbon quantum dot.

The weight ratio of the carbon quantum dot, bonded to the Ce6, to the hyaluronic acid or the derivative thereof may range from 4:1 to 2:1.

The present invention also provides a method of manufacturing a complex, the method including (a) covalently bonding a carbon nanomaterial, bonded to a photosensitizer, to a hyaluronic acid or a derivative thereof.

The covalently bonding the carbon nanomaterial may include (a′) mixing or dissolving the hyaluronic acid or the derivative thereof and the carbon nanomaterial bonded to the photosensitizer, and then adding 1-(3-dimethylaminopropyl)-3-ethylcarbodiimide hydrochloride (EDC) as a catalyst to perform a reaction.

The method may further include (b) removing the EDC after the step (a′).

The reaction may be performed under a lightless condition.

The present invention also provides a composition for remedying or treating a retinal disease, including the complex for remedying or treating the retinal disease as an effective component.

A complex for remedying or treating a retinal disease according to the present invention includes a hyaluronic acid-carbon nanomaterial-photosensitizer complex, thus selectively preventing the formation of active oxygen in retinal pigment epithelium cells over a relatively long period of time. Accordingly, the complex has excellent therapeutic efficacy and easily infiltrates into cells.

Further, the present invention provides a composition for remedying or treating a retinal disease including the hyaluronic acid-carbon nanomaterial-photosensitizer complex.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a view schematically showing the administration of a complex for remedying or treating a retinal disease of the present invention into an eye;

FIG. 2 shows the result of analysis of the probability of prevention of active oxygen formation by a hyaluronic acid-carbon quantum dot-Ce6 complex (Cdot-Ce6-HA), manufactured in Example 1, a carbon quantum dot-Ce6 complex (Cdot-Ce6), and Ce6 using DPPH (2,2-diphenyl-1-picrylhydrazyl);

FIG. 3 shows the result obtained by treating a retinal pigment epithelium cell using the hyaluronic acid-carbon quantum dot-Ce6 complex, manufactured in Example 1, the carbon quantum dot-Ce6 complex, and Ce6, performing incubation for 24 hours, and checking cytotoxicity using an MTT assay;

FIG. 4 shows the result obtained by treating the retinal pigment epithelium cell using 0, 2.5, and 5 mM sodium iodate, performing incubation for 24 hours, and comparing cell viabilities;

FIG. 5 shows the result obtained by treating the retinal pigment epithelium cell using the hyaluronic acid-carbon quantum dot-Ce6 complex manufactured in Example 1, the carbon quantum dot-Ce6 complex, and Ce6, further treating the resultant retinal pigment epithelium cell using 5 mM sodium iodate, performing incubation for 24 hours, and comparing cell viabilities using an MTT assay;

FIG. 6 shows the result obtained by treating the retinal pigment epithelium cell using the hyaluronic acid-carbon quantum dot-Ce6 complex manufactured in Example 1, the carbon quantum dot-Ce6 complex, and Ce6 for 2 hours, further treating the resultant retinal pigment epithelium cell using sodium iodate, performing incubation for 24 hours, and measuring the change in active oxygen using a DCF-DA (2′,7′-dichlorofluorescin diacetate) assay;

FIG. 7 shows the result obtained by treating the retinal pigment epithelium cell using the hyaluronic acid-carbon quantum dot-Ce6 complex manufactured in Example 1, the carbon quantum dot-Ce6 complex, and Ce6, further treating the resultant retinal pigment epithelium cell using sodium iodate, performing incubation for 24 hours, and measuring the concentration of glutathione using a GSH-400 kit;

FIG. 8 shows the result obtained by treating the retinal pigment epithelium cell using various processes, performing incubation for 24 hours, and checking cell uptake using a confocal scanning laser microscope;

FIG. 9 shows the result obtained by treating the retinal pigment epithelium cell using various processes, performing incubation for 24 hours, performing an F-actin assay, and checking the shape and the concentration of actin using the confocal scanning laser microscope;

FIG. 10 shows the result obtained by treating the retinal pigment epithelium cell using various processes, performing incubation for 24 hours, treating the resultant retinal pigment epithelium cell using a CellROX™ green agent, and checking the treated cell using the confocal scanning laser microscope;

FIGS. 11(a) and 11(b) show the result obtained by delivering a PBS (phosphate buffer saline, none), Ce6, and the hyaluronic acid-carbon quantum dot-Ce6 complex, manufactured in Example 1, into the retina of a normal rabbit eye using intravitreous injection, and checking the retinal pigment epithelium cell uptake and the time remaining therein using the confocal scanning laser microscope; and

FIGS. 12(a), 12(b) and 12(c) show the result obtained by delivering the PBS, Ce6, and the hyaluronic acid-carbon quantum dot-Ce6 complex, manufactured in Example 1, into the normal rabbit eye using intravitreous injection, and checking the change in electric signal using electroretinography (ERG).

### DESCRIPTION OF THE PREFERRED EMBODIMENTS

The present invention will be described more fully hereinafter with reference to the accompanying drawings, in which exemplary embodiments of the invention are shown so as to be easily understood by a person with ordinary skill in the art.

However, the following description does not limit the present invention to the specific embodiments, and descriptions of known techniques, even if they are pertinent to the present invention, are considered unnecessary and may be omitted insofar as they would make the characteristics of the invention unclear.

The terms used herein are merely intended to explain specific examples and not to limit the present invention. Unless otherwise stated, the singular expression includes a plural expression. In this application, the terms “include” or “have” are used to designate the presence of features, numbers, steps, operations, elements, or combinations thereof described in the specification, and should be understood as not excluding the presence or additional possibility of one or more different features, numbers, steps, operations, elements, or combinations thereof.

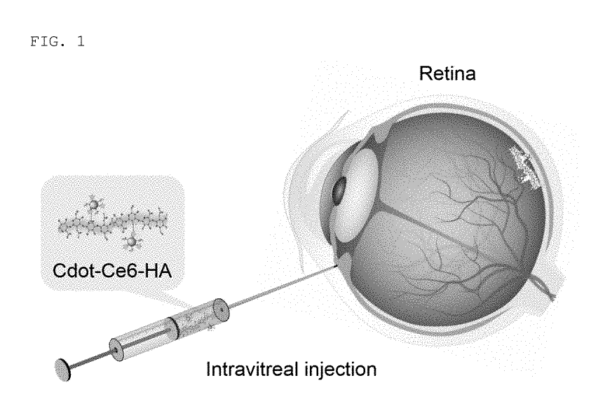

FIG. 1 is a view schematically showing the administration of a complex for remedying or treating a retinal disease of the present invention into an eye. The complex for remedying or treating the retinal disease is a hyaluronic acid-carbon quantum dot-Ce6 complex (Cdot-Ce6-HA) including a hyaluronic acid, a carbon quantum dot, and chlorine e6 (Ce6) bonded to each other, and is administered using intravitreous injection, without being limited thereto.

Hereinafter, the complex for remedying or treating the retinal disease according to the present invention will be described in detail with reference to FIG. 1. However, this is intended to illustrate the present invention but does not limit the present invention, and the present invention is defined by the scope of the claims as will be described later.

The complex for remedying or treating the retinal disease according to the present invention may include a hyaluronic acid or a derivative thereof, a carbon nanomaterial covalently bonded to the hyaluronic acid or the derivative thereof, and a photosensitizer bonded to the carbon nanomaterial.

In the present specification, the bonding may be chemical or physical bonding, and preferably chemical bonding. Specifically, the bonding may be covalent bonding, ionic bonding, or coordinate bonding, and preferably covalent bonding.

The complex for remedying or treating the retinal disease may prevent active oxygen from being formed, and may preferably prevent active oxygen from being formed in a retinal pigment epithelium cell or a retinal vessel.

Examples of the retinal disease include retinitis pigmentosa, dry macular degeneration, and nyctalopia, but are not limited thereto. The retinal disease may include any retinal disease induced by degeneration of retinal pigment epithelium cells owing to active oxygen.

It is preferable that treatment using the complex for remedying or treating the retinal disease be performed under a lightless condition. The complex for remedying or treating the retinal disease may be administered using a method such as intravitreous injection, administration of eye drops, or intravenous injection.

The derivative of the hyaluronic acid may be a hyaluronic acid substituted with cystamine having the structure of the following Chemical Formula 1.

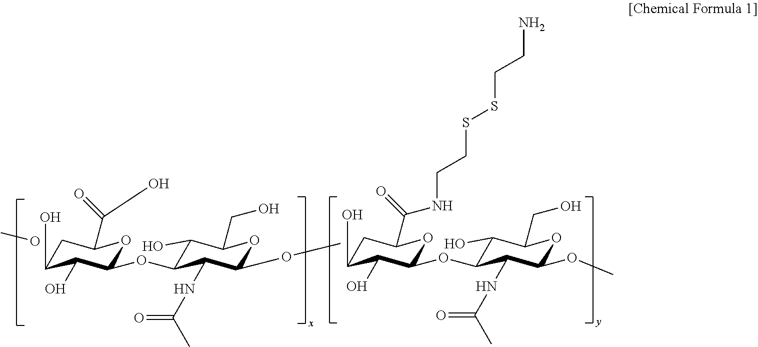

In Chemical Formula 1, x and y are each independently an integer selected from 16 to 2,500.

Preferably, x and y of Chemical Formula 1 may be determined depending on a substitution ratio. For example, when the substitution ratio is 30%, 20%, and 10% respectively, the ratio of x and y may be an integer ratio of 7:3, 8:2, and 9:1.

Substitution with the cystamine may be performed at the substitution ratio of 10 to 50%, preferably 10 to 30%, and more preferably 10%, based on the hyaluronic acid.

Examples of the carbon nanomaterial may include a carbon quantum dot, a carbon nanotube, graphene oxide, graphene, and fullerene, and the carbon nanomaterial may preferably be the carbon quantum dot.

Examples of the photosensitizer may include chlorine e6 (Ce6), a porphyrin-based photosensitizer, and a non-porphyrin-based photosensitizer, and the photosensitizer may be preferably chlorine e6.

The photosensitizer is a chemical material that generates a strong fluorescent signal owing to light having a predetermined wavelength or generating both the fluorescent signal and the reactive oxygen species. When the distance between the carbon nanomaterials has a predetermined small value, the generation of the fluorescent signal and/or the reactive oxygen species may be prevented by energy transfer between the carbon nanomaterials.

1 to 2 parts by weight of Ce6 may be bonded to 1 part by weight of the carbon quantum dot.

The weight ratio of the carbon quantum dot bonded to Ce6 and the hyaluronic acid or the derivative thereof may be 4:1 to 2:1, and preferably 3:1.

Hereinafter, a method of manufacturing the complex according to the present invention will be described.

The carbon nanomaterial bonded to the photosensitizer may be covalently bonded to the hyaluronic acid or the derivative thereof, thereby manufacturing the complex according to the present invention (step a).

The covalently bonding the carbon nanomaterial may include mixing or dissolving the hyaluronic acid or the derivative thereof and the carbon nanomaterial bonded to the photosensitizer, and then adding 1-(3-dimethylaminopropyl)-3-ethylcarbodiimide hydrochloride (EDC) as a catalyst to perform a reaction (step (a′)).

Preferably, the derivative of the hyaluronic acid, which is obtained by substituting the hyaluronic acid with cystamine, may be mixed with the carbon quantum dot-Ce6 complex bonded to Ce6 as the photosensitizer to thus perform an amide reaction of one remaining amine group, which is not bonded to the hyaluronic acid, of cystamine groups and the carboxylic group of Ce6 of the carbon quantum dot-Ce6 complex bonded to Ce6 as the photosensitizer, thereby manufacturing the complex according to the present invention.

The reaction may be performed under a lightless condition.

The method may further include removing the EDC after the step (a′) (step (b)).

Further, the present invention may provide a composition for remedying or treating a retinal disease, including a complex for remedying or treating a retinal disease.

The photodynamic therapy composition may include a hyaluronic acid-carbon nanomaterial complex in a pharmaceutically effective amount and a pharmaceutically allowable carrier. The carrier may be a diluent, and the composition may further include an adjuvant such as a preservative, a wetting agent, an emulsifier, and a dispersant. The pharmaceutical composition may be formulated so as to match the intended administration path.

### EXAMPLE

A better understanding of the present invention may be obtained through the following examples which are set forth to illustrate, but are not to be construed to limit the present invention.

### Preparation Example 1: Manufacture of Hyaluronic Acid Substituted with Diaminohexane

A molar amount of diaminohexane equal to 20 times the molar amount of a hyaluronic acid monomer or the larger molar amount of diaminohexane was added to 5 mg/1 mL of an aqueous solution including 210 kDa of a hyaluronic acid. The pH was reduced to 5.5, a reaction was performed for 10 min using EDC and sulfo-NHS (sulfo-N-hydroxysuccinimide), the pH was increased to 7.4 to terminate the reaction, and the resultant material was purified using dialysis and freeze-dried, thereby obtaining the hyaluronic acid substituted with diaminohexane.

### Preparation Example 2: Manufacture of Carbon Quantum Dot-Ce6 Complex

NHS and EDC were added in the same molar number to a solution of 1 mg of Ce6/1 mL DMSO to activate a carboxylic group for 30 min, mixed with a solution of 2 mg of a carbon quantum dot/1 mL of a phosphate buffer to perform a reaction in a dark room for 12 hours, purified, and freeze-dried, thereby obtaining the carbon quantum dot-Ce6 complex.

### Example 1: Manufacture of Hyaluronic Acid-Carbon Quantum Dot-Ce6 Complex

The hyaluronic acid, substituted with diaminohexane, manufactured in Preparation Example 1 and the carbon quantum dot-Ce6 complex manufactured in Preparation Example 2 were mixed at a ratio of 5:2, and were reacted in a dark room using EDC for 18 hours to thus manufacture a hyaluronic acid-carbon quantum dot-Ce6 complex. Subsequently, the complex was purified in a phosphate buffer for one day and in distilled water for one day in order to remove EDC, which was a catalyst.

### Test Example

### Test Example 1: Analysis of Prevention of Active Oxygen

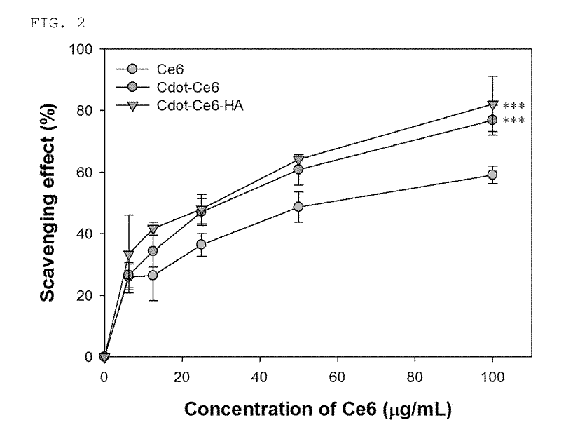

The probability of suppression of active oxygen by a hyaluronic acid-carbon quantum dot-Ce6 complex (Cdot-Ce6-HA), manufactured in Example 1, a carbon quantum dot-Ce6 complex (Cdot-Ce6), manufactured in Preparation Example 2, and Ce6 (Santa Cruz Biotechnology) was analyzed using DPPH (2,2-diphenyl-1-picrylhydrazyl), and the results are shown in FIG. 2.

The hyaluronic acid-carbon quantum dot-Ce6 complex manufactured in Example 1, the carbon quantum dot-Ce6 complex manufactured in Preparation Example 2, and Ce6 were tested in an aqueous solution at concentrations of 0, 20, 40, 60, 80, and 100 μg/mL based on the amount of Ce6.

From FIG. 2, it was confirmed that the active oxygen suppression ability of the hyaluronic acid-carbon quantum dot-Ce6 complex was 80% while that of Ce6 was 60%. Further, based on the fact that the active oxygen suppression ability of the hyaluronic acid-carbon quantum dot-Ce6 complex is slightly higher than that of the carbon quantum dot-Ce6 complex, the hyaluronic acid is considered to have a satisfactory ability to suppress active oxygen.

### Test Example 2: Analysis of Cytotoxicity

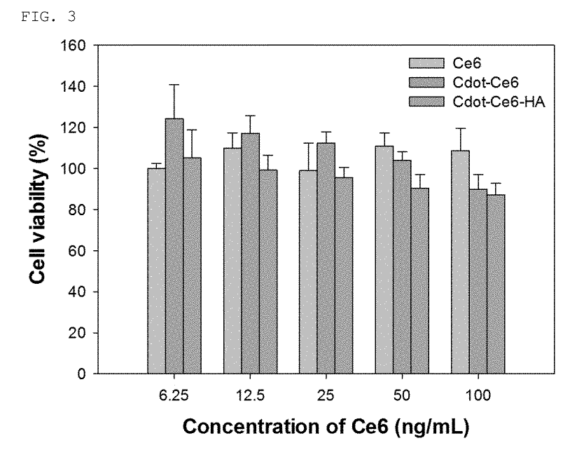

A retinal pigment epithelium cell (ARPE-19, ATCC® CRL-2302™) was treated using the hyaluronic acid-carbon quantum dot-Ce6 complex (Cdot-Ce6-HA) manufactured in Example 1, the carbon quantum dot-Ce6 complex (Cdot-Ce6) manufactured in Preparation Example 2, and Ce6, was incubated for 24 hours, and was subjected to an MTT assay to check cytotoxicity, and the results are shown in FIG. 3.

The hyaluronic acid-carbon quantum dot-Ce6 complex manufactured in Example 1, the carbon quantum dot-Ce6 complex manufactured in Preparation Example 2, and Ce6 were tested in an aqueous solution at concentrations of 6.25, 12.5, 25, 50, and 100 ng/mL based on the amount of Ce6.

Referring to FIG. 3, the hyaluronic acid-carbon quantum dot-Ce6 complex manufactured in Example 1 does not exhibit toxicity until the concentration of Ce6 reaches 100 ng/mL, but exhibits toxicity relatively higher than those of Ce6 and the carbon quantum dot-Ce6 complex manufactured in Preparation Example 2.

This is caused by the difference in uptake between the hyaluronic acid-carbon quantum dot-Ce6 complex, including the hyaluronic acid and other materials. It is considered that the hyaluronic acid-carbon quantum dot-Ce6 complex according to the present invention selectively acts on a retinal pigment epithelium cell to thus effectively exhibit a treatment effect.

### Test Example 3: MTT Assay of Retinal Pigment Epithelium Cell Treated Using Sodium Iodate

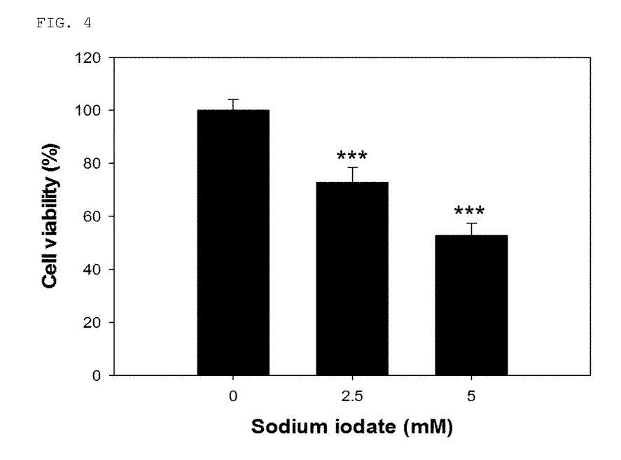

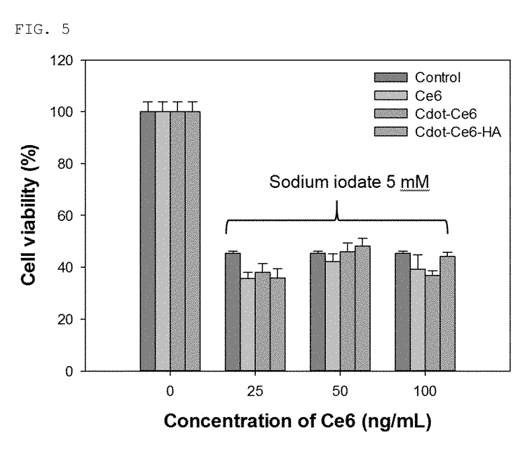

A retinal pigment epithelium cell was treated using 0, 2.5, and 5 mM sodium iodate (NaIO₃) and was incubated for 24 hours, cell viabilities thereof were compared, and the results are shown in FIG. 4. A retinal pigment epithelium cell was treated using the hyaluronic acid-carbon quantum dot-Ce6 complex (Cdot-Ce6-HA) manufactured in Example 1, the carbon quantum dot-Ce6 complex (Cdot-Ce6) manufactured in Preparation Example 2, and Ce6, was further treated using 5 mM sodium iodate, was incubated for 24 hours, and was subjected to an MTT assay to thus obtain cell viability, and the results are shown in FIG. 5.

The hyaluronic acid-carbon quantum dot-Ce6 complex manufactured in Example 1, the carbon quantum dot-Ce6 complex manufactured in Preparation Example 2, and Ce6 were tested in an aqueous solution at concentrations of 0, 25, 50, and 100 ng/mL based on the amount of Ce6.

Referring to FIG. 4, the viability was reduced as the concentration of sodium iodate was increased. Therefore, it could be confirmed that sodium iodate for inducing retinitis pigmentosa in an animal model degenerated the retina cell.

Referring to FIG. 5, the viability of the retinal pigment epithelium cell treated using 5 mM sodium iodate was reduced to 40%, and the viability of the cell pre-treated using Ce6 and the carbon quantum dot-Ce6 complex was lower than that of the retinal pigment epithelium cell treated using sodium iodate. However, in the case where pre-treatment was performed using the hyaluronic acid-carbon quantum dot-Ce6 complex, when the concentration of Ce6 was 50 ng/mL, the hyaluronic acid-carbon quantum dot-Ce6 complex more strongly suppressed the degeneration of retinal pigment epithelium cells than other materials.

### Test Example 4: DCF-DA Assay of Retinal Pigment Epithelium Cell Treated Using Sodium Iodate

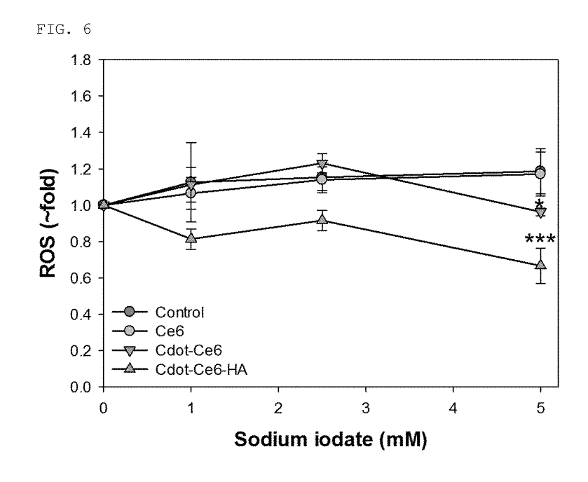

A retinal pigment epithelium cell was treated using the hyaluronic acid-carbon quantum dot-Ce6 complex (Cdot-Ce6-HA) manufactured in Example 1, the carbon quantum dot-Ce6 complex (Cdot-Ce6) manufactured in Preparation Example 2, and Ce6 for 2 hours, was further treated using sodium iodate, and was incubated for 24 hours, and the change in active oxygen was measured using a DCF-DA (2′,7′-dichlorofluorescin diacetate) assay. The results are shown in FIG. 6.

The retinal pigment epithelium cell was treated using 0, 1, 2, 3, 4, and 5 mM of sodium iodate. The hyaluronic acid-carbon quantum dot-Ce6 complex manufactured in Example 1, the carbon quantum dot-Ce6 complex manufactured in Preparation Example 2, and Ce6 were tested in an aqueous solution at a concentration of 100 ng/mL based on the amount of Ce6.

Referring to FIG. 6, when the retinal pigment epithelium cell was pre-treated using the hyaluronic acid-carbon quantum dot-Ce6 complex, treated using 5 mM sodium iodate, and incubated for 24 h, the concentration of active oxygen was reduced by 30% compared to the concentration of active oxygen when the retinal pigment epithelium cell was treated using only 5 mM sodium iodate without any other treatment (control).

Therefore, it can be seen that the hyaluronic acid-carbon quantum dot-Ce6 complex prevents the formation of active oxygen by sodium iodate.

### Test Example 5: Analysis of Concentration of Glutathione of Retinal Pigment Epithelium Cell Treated Using Sodium Iodate

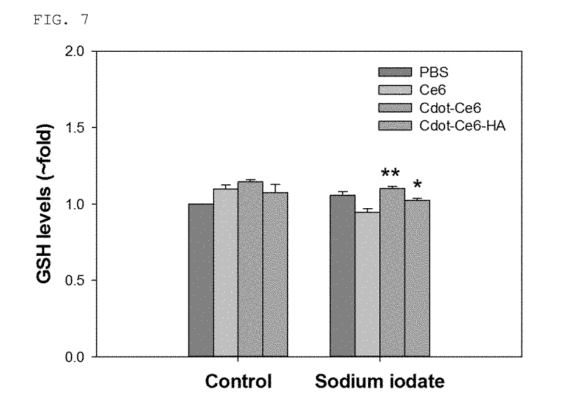

A retinal pigment epithelium cell was treated using the hyaluronic acid-carbon quantum dot-Ce6 complex (Cdot-Ce6-HA) manufactured in Example 1, a carbon quantum dot-Ce6 complex (Cdot-Ce6), and Ce6, was further treated using 5 mM sodium iodate, and was incubated for 24 hours, and the concentration of glutathione was measured using a GSH-400 kit. The results are shown in FIG. 7.

The hyaluronic acid-carbon quantum dot-Ce6 complex manufactured in Example 1, the carbon quantum dot-Ce6 complex manufactured in Preparation Example 2, and Ce6 were tested in an aqueous solution at a concentration of 100 ng/mL based on the amount of Ce6.

Referring to FIG. 7, the retinal pigment epithelium cell not treated using sodium iodate (control) and the retinal pigment epithelium cell treated using sodium iodate (sodium iodate) did not differ in terms of the concentration of glutathione. Therefore, it is considered that active oxygen does not have any correlation with glutathione.

### Test Example 6: Checking of Cell Uptake

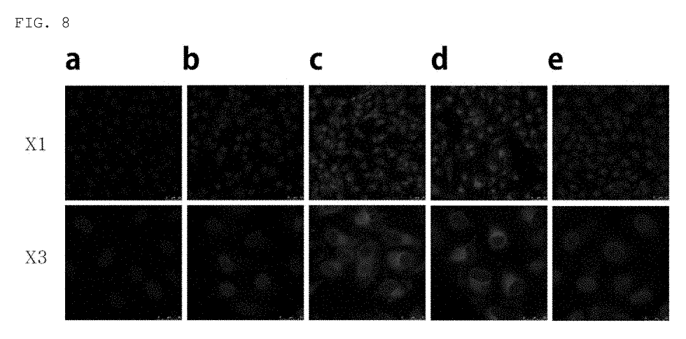

A retinal pigment epithelium cell was treated using various processes and was incubated for 24 hours, and cell uptake was checked using a confocal scanning laser microscope. The results are shown in FIG. 8. FIGS. 8a, 8b, 8c, 8d, and 8e show the cell that was not treated, the cell treated using Ce6, the cell treated using the carbon quantum dot-Ce6 complex, the cell treated using the hyaluronic acid-carbon quantum dot-Ce6 complex, and the retinal pigment epithelium cell when the receptor of the hyaluronic acid is blocked, respectively. The upper line shows images magnified by a factor of 1×, and the lower line shows images magnified by a factor of 3×.

The hyaluronic acid-carbon quantum dot-Ce6 complex manufactured in Example 1, the carbon quantum dot-Ce6 complex manufactured in Preparation Example 2, and Ce6 were tested in an aqueous solution at a concentration of 100 ng/mL based on the amount of Ce6.

Referring to FIG. 8, the hyaluronic acid-carbon quantum dot-Ce6 complex manufactured in Example 1 exhibited a cell uptake ratio that was higher than that of Ce6 and the carbon quantum dot-Ce6 complex manufactured in Preparation Example 2. Further, when treatment was performed using the hyaluronic acid-carbon quantum dot-Ce6 complex after pre-treatment using the hyaluronic acid at a high concentration, the cell uptake was reduced.

Therefore, it can be confirmed that the retinal pigment epithelium cell has the receptor of the hyaluronic acid, thus ensuring the high cell uptake of the hyaluronic acid-carbon quantum dot-Ce6 complex.

### Test Example 7: Checking of Cell Stress and Change of Actin Due to Sodium Iodate

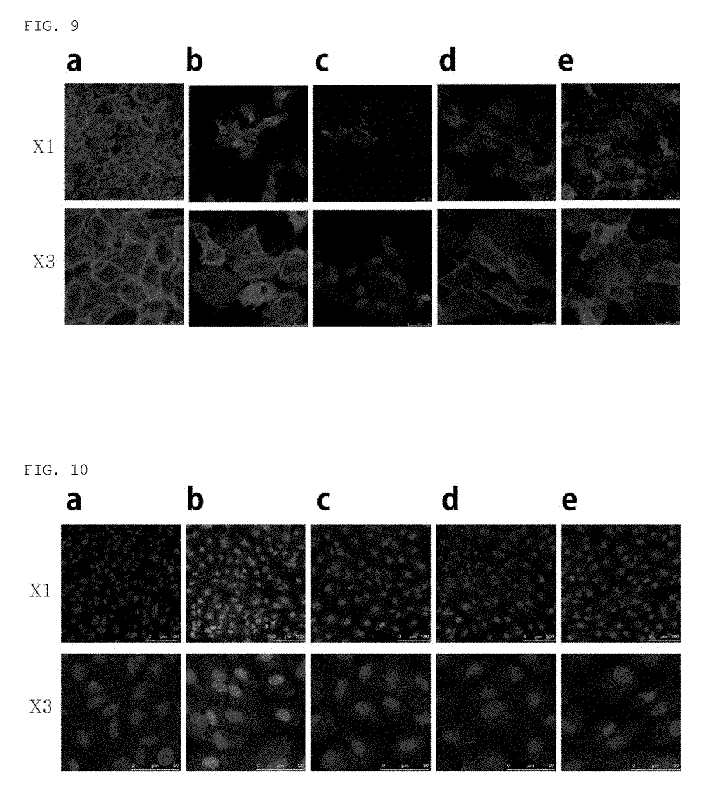

A retinal pigment epithelium cell was treated using various processes, was incubated for 24 hours, and was subjected to an F-actin assay, and the shape and the concentration of actin were checked using a confocal scanning laser microscope. The results are shown in FIG. 9. FIGS. 9a, 9b, 9c, 9d, and 9e show the cell that was not treated, the cell treated using only sodium iodate, the cell treated using Ce6, the cell treated using the carbon quantum dot-Ce6 complex, and the cell treated using the hyaluronic acid-carbon quantum dot-Ce6 complex and subsequently sodium iodate, respectively. The upper line shows images magnified by a factor of 1×, and the lower line shows images magnified by a factor of 3×.

The hyaluronic acid-carbon quantum dot-Ce6 complex manufactured in Example 1 was tested in an aqueous solution at a concentration of 100 ng/mL based on the amount of Ce6.

From FIG. 9, it could be seen that actin was almost completely removed from the retinal pigment epithelium cell treated using sodium iodate, the cell boundary vanished, and the number of cells was significantly reduced. However, in the case of the retinal pigment epithelium cell that was pre-treated using the hyaluronic acid-carbon quantum dot-Ce6 complex, treated using sodium iodate, and then incubated, the number of cells was seldom reduced, and a predetermined amount of actin remained in the cells, compared to normal cells.

Therefore, it is considered that the hyaluronic acid-carbon quantum dot-Ce6 complex suppresses the deformation and degeneration of retinal pigment epithelium cells by sodium iodate.

### Test Example 8: Checking of Suppression of Active Oxygen by Hyaluronic Acid-Carbon Quantum Dot-Ce6 Complex

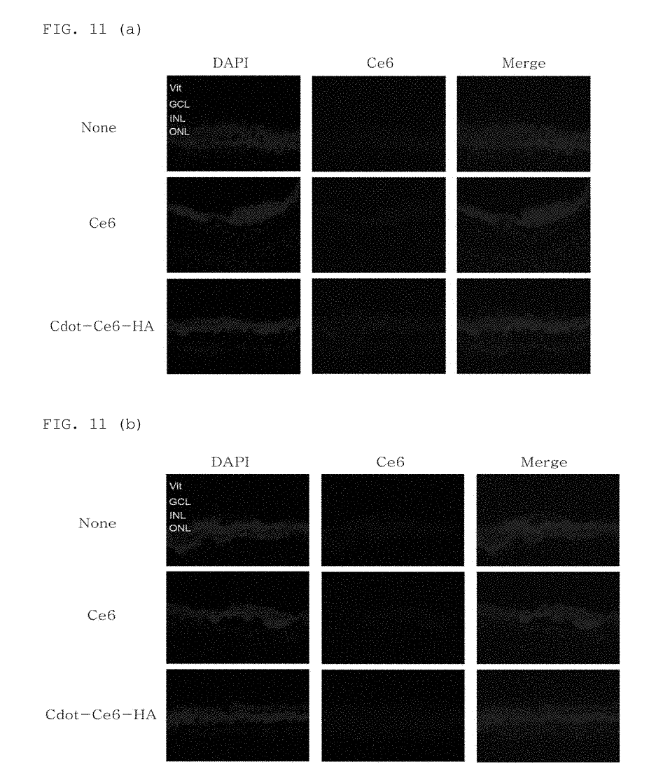

A retinal pigment epithelium cell was treated using various processes, was incubated for 24 hours, was treated using a CellROX™ green agent, and was observed using a confocal scanning laser microscope. The results are shown in FIG. 10. FIGS. 10a, 10b, 10c, 10d, and 10e show the cell that was not treated, the cell treated using sodium iodate, the cell treated using Ce6, the cell treated using the carbon quantum dot-Ce6 complex, and the cell treated using the hyaluronic acid-carbon quantum dot-Ce6 complex and, subsequently, 5 mM sodium iodate, respectively. The upper line shows images magnified by a factor of 1×, and the lower line shows images magnified by a factor of 3×.

The hyaluronic acid-carbon quantum dot-Ce6 complex manufactured in Example 1 was tested in an aqueous solution at a concentration of 100 ng/mL based on the amount of Ce6.

Referring to FIG. 10, in the retina cell treated using only sodium iodate (FIG. 10b), active oxygen was formed in a large amount and reacted with a CellROX™ green agent, thus emitting strong blue fluorescent light. The retinal pigment epithelium cell pre-treated using the hyaluronic acid-carbon quantum dot-Ce6 complex (FIG. 10e) prevented active oxygen from being formed, and accordingly, blue fluorescent light was seldom exhibited.

Therefore, it is considered that the hyaluronic acid-carbon quantum dot-Ce6 complex prevents the degeneration of retinal pigment epithelium cells owing to active oxygen.

### Test Example 9: Checking of Uptake and Time of Hyaluronic Acid-Carbon Quantum Dot-Ce6 Complex Remaining in Retinal Pigment Epithelium Cell of Rabbit (In Vivo)

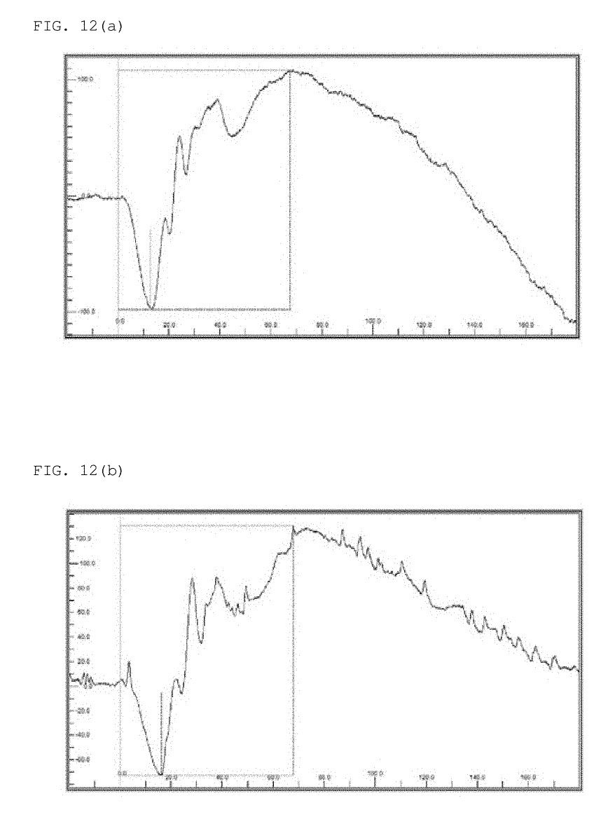

A PBS (phosphate buffer saline, none), Ce6, and the hyaluronic acid-carbon quantum dot-Ce6 complex (Cdot-Ce6-HA) manufactured in Example 1 were delivered into the retina of a normal rabbit eye using intravitreous injection, and the uptake and the time each remained in a retinal pigment epithelium cell were checked using a confocal scanning laser microscope. The results are shown in FIG. 11. FIGS. 11a and 11b show retinal tissue 6 hours after intravitreous injection and retinal tissue 24 hours after intravitreous injection, respectively. DAPI is a dyeing material used to dye the nucleus of retinal tissue and has a fluorescent blue color, and Ce6 has a fluorescent red color in order to observe the Ce6 material administered using intravitreous injection. A Merge image is obtained by combining DAPI and Ce6 images.

The hyaluronic acid-carbon quantum dot-Ce6 complex manufactured in Example 1 and Ce6 were tested in an aqueous solution at a concentration of 100 ng/mL based on the amount of Ce6.

From FIG. 11, it could be confirmed that when the hyaluronic acid-carbon quantum dot-Ce6 complex manufactured in Example 1 was administered using intravitreous injection, the uptake ratio of the complex in the retinal pigment epithelium cell was high and the complex remained therein for 24 hours.

### Test Example 10: Checking of Change in Electric Signal of Retina after Hyaluronic Acid-Carbon Quantum Dot-Ce6 Complex is Administered into Rabbit Using Intravitreous Injection

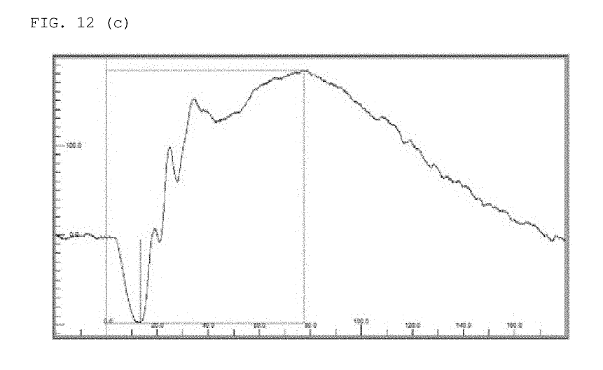

A PBS (none), Ce6, and the hyaluronic acid-carbon quantum dot-Ce6 complex (Cdot-Ce6-HA) manufactured in Example 1 were delivered into a normal rabbit eye using intravitreous injection in respective amounts of 5 μg based on 1 kg of the rabbit weight (the concentration of the aqueous solution was 100 ng/mL), and a change in an electric signal was checked using electroretinography (ERG). The results are shown in FIG. 12. FIGS. 12a, 12b, and 12c are electroretinographs of a normal retina, the retina treated using Ce6, and the retina treated using the hyaluronic acid-carbon quantum dot-Ce6 complex.

The hyaluronic acid-carbon quantum dot-Ce6 complex manufactured in Example 1 and Ce6 were tested in an aqueous solution at a concentration of 100 ng/mL based on the amount of Ce6.

Referring to FIG. 12, electric signals of the eye treated using PBS and the eye treated using the hyaluronic acid-carbon quantum dot-Ce6 complex manufactured in Example 1 were not different. This means that there is no functional degeneration or loss.

Therefore, it can be seen that the hyaluronic acid-carbon quantum dot-Ce6 complex manufactured in Example 1 is not toxic to the normal rabbit retina.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A complex for remedying or treating a retinal disease, comprising:
a hyaluronic acid or a derivative thereof;
a carbon nanomaterial covalently bonded to the hyaluronic acid or the derivative thereof; and
a photosensitizer bonded to the carbon nanomaterial.

2. The complex of claim 1, wherein the complex for remedying or treating the retinal disease prevents active oxygen from being formed.

3. The complex of claim 2, wherein the complex for remedying or treating the retinal disease prevents the active oxygen from being formed in a retinal pigment epithelium cell or a retinal vessel.

4. The complex of claim 3, wherein the retinal disease includes one or more selected from retinitis pigmentosa, dry macular degeneration, and nyctalopia.

5. The complex of claim 4, wherein treatment using the complex for remedying or treating the retinal disease is performed under a lightless condition.

6. The complex of claim 5, wherein the complex for remedying or treating the retinal disease is administered using one or more selected from intravitreous injection, administration of eye drops, and intravenous injection.

7. The complex of claim 1, wherein the derivative of the hyaluronic acid is the hyaluronic acid substituted with cystamine having a structure of the following Chemical Formula 1: 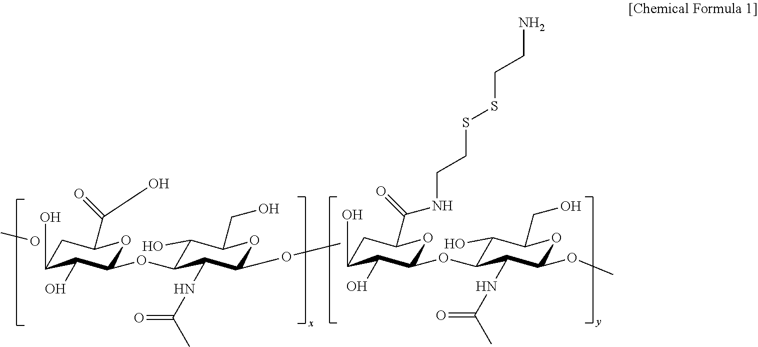
where x and y are each independently an integer selected from 16 to 2,500.

8. The complex of claim 7, wherein substitution with the cystamine is performed at a substitution ratio of 10 to 30% based on the hyaluronic acid.

9. The complex of claim 1, wherein the carbon nanomaterial includes one or more selected from a carbon quantum dot, a carbon nanotube, graphene oxide, graphene, and fullerene.

10. The complex of claim 9, wherein a photosensitizer includes one or more selected from chlorine e6 (Ce6), a porphyrin-based photosensitizer, and a non-porphyrin-based photosensitizer.

11. The complex of claim 9, wherein the carbon nanomaterial is the carbon quantum dot and the photosensitizer is the Ce6.

12. The complex of claim 11, wherein 1 to 2 parts by weight of the Ce6 is bonded to 1 part by weight of the carbon quantum dot.

13. The complex of claim 12, wherein a weight ratio of the carbon quantum dot bonded to the Ce6 and a hyaluronic acid or a derivative thereof is 4:1 to 2:1.

14. A method of manufacturing a complex, the method comprising:
(a) covalently bonding a carbon nanomaterial, bonded to a photosensitizer, to a hyaluronic acid or a derivative thereof.

15. The method of claim 14, wherein the covalently bonding the carbon nanomaterial includes (a′) mixing or dissolving the hyaluronic acid or the derivative thereof and the carbon nanomaterial bonded to the photosensitizer, and then adding 1-(3-dimethylaminopropyl)-3-ethylcarbodiimide hydrochloride (EDC) as a catalyst to perform a reaction.

16. The method of claim 15, further comprising:
(b) removing the EDC after the step (a′).

17. The method of claim 15, wherein the reaction is performed under a lightless condition.

18. A composition for remedying or treating a retinal disease, comprising:
the complex of claim 1 as an effective component.
